(19) United States
(12) Patent Application Publication
(10) Pub. No.: US 2024/0099875 A1
(43) Pub. Date: Mar. 28, 2024

(54) AN OSTOMY COLLECTION DEVICE

(71) Applicant: Salts Healthcare Limited, Birmingham (GB)

(72) Inventors: Marcus Allen, Birmingham (GB); Mark Heynes, Beaconside (GB); Phillip Sampey, Beconside (GB)

(21) Appl. No.: 18/275,592

(22) PCT Filed: Feb. 3, 2022
(86) PCT No.: PCT/GB2022/050291

§ 371 Date: Aug. 2, 2023

(30) Foreign Application Priority Data
Feb. 5, 2021 (GB) 2101652.2

(51) Int. Cl. A61F 5/441; A61F 5/44; A61F 5/445

(52) U.S. Cl. CPC A61F 5/441; A61F 5/4407; A61F 5/445

(57) ABSTRACT

An ostomy collection device (10) including: a housing (12) which defines a collecting volume for receiving and storing waste from an ostomy appliance, an inlet (30) for fluidly connecting to an outlet of an ostomy appliance so that waste from the ostomy appliance flows into the collecting volume, an outlet (32) for emptying the contents of the collecting volume, and a lid portion (40) which is slidable between a first position, in which the outlet (32) is open and waste can be emptied from the collecting volume and a second position, in which the outlet (32) is closed/covered.

Embodiments of the present invention relate to an ostomy collection device.

Ostomy appliances are well known. Typically, a person having a stoma uses an ostomy appliance to collect waste from their stoma. The ostomy appliances collects a volume of waste and, once full or nearing full, the user either changes the appliance for a fresh one or empties the waste from the existing appliance.

This process works well while the user is able to visit a suitable space for changing/emptying their ostomy appliance—e.g. a bathroom or toilet. However, there may be times when the user does not wish to empty or change their appliance as often, for example, when travelling or while asleep.

In such times, it would be beneficial to provide an additional collecting volume that is connected to the existing ostomy appliance, so that the appliance does not need to be emptied as often. This functionality is currently provided by a flexible bag which is connected to the ostomy appliance.

Alternative systems are sought for ostomy appliance users. Embodiments of the present invention seeks to alleviate one or more drawbacks of the prior art.

According to an aspect of the invention, we provide an ostomy collection device including: a housing which defines a collecting volume for receiving and storing waste from an ostomy appliance, an inlet for fluidly connecting to an outlet of an ostomy appliance so that waste from the ostomy appliance flows into the collecting volume, an outlet for emptying the contents of the collecting volume, and a lid portion which is slidable between a first position, in which the outlet is open and waste can be emptied from the collecting volume and a second position, in which the outlet is closed/covered.

Further optional features of the invention are set out in the appended claims.

Embodiments of the present invention are described, by way of example only, with reference to the accompanying drawings, in which:

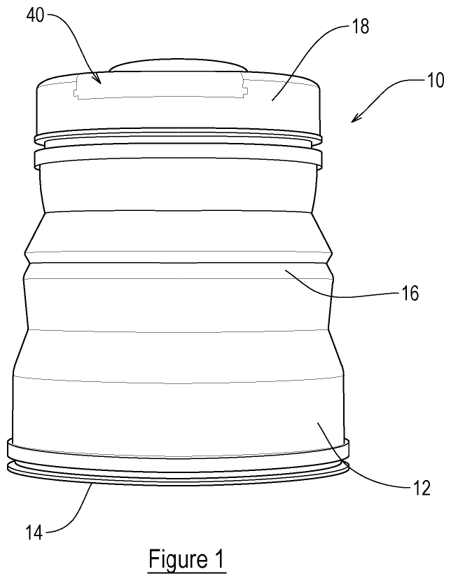

FIG. 1 is a side view of an ostomy collection device,

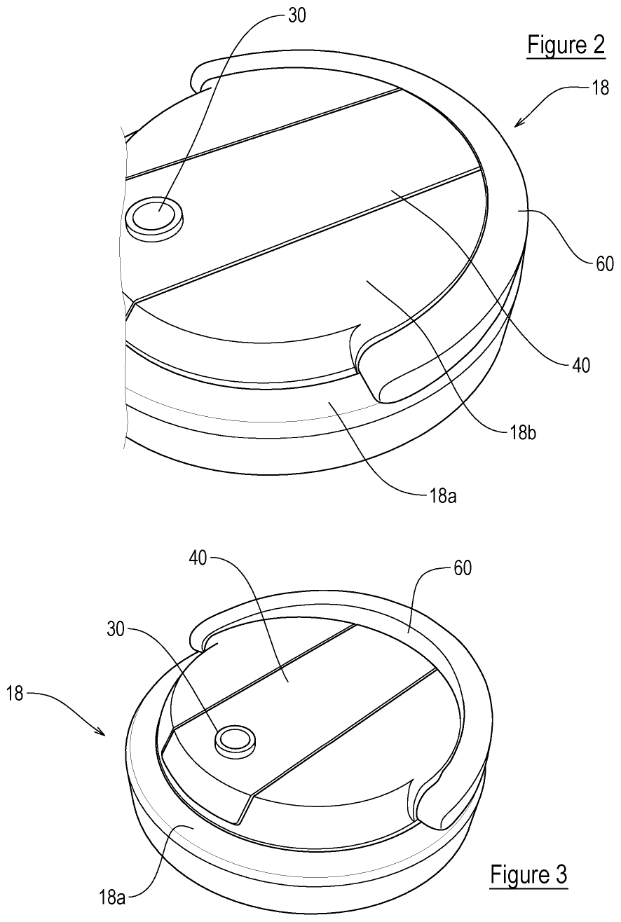

FIG. 2 is a perspective view of a part of the ostomy collection device,

FIG. 3 is a view of the part of the ostomy collection device,

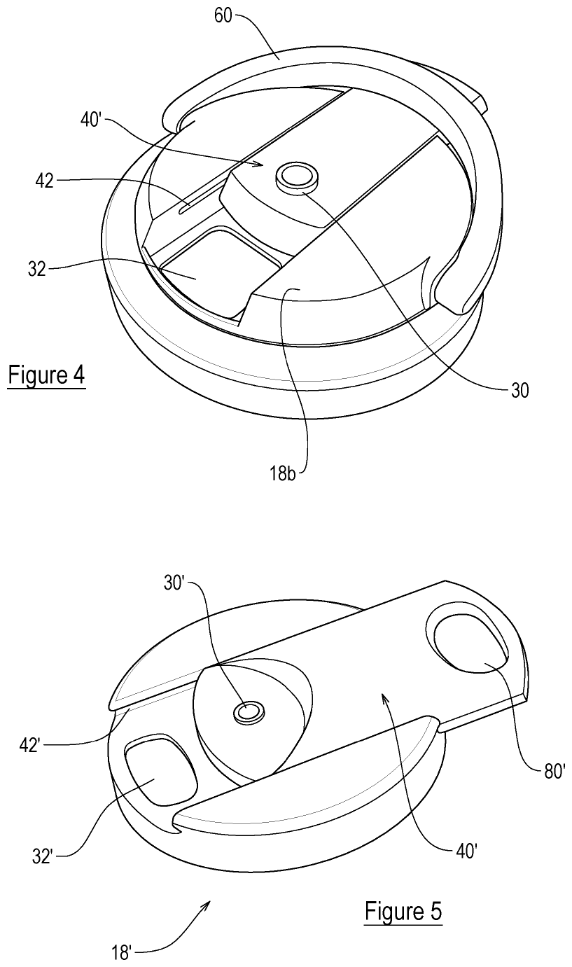

FIG. 4 is a view of the part of the ostomy collection device, and

FIG. 5 is a view of a second embodiment of the ostomy collection device.

An ostomy collection device 10 is shown in FIG. 1. The ostomy collection device 10 is configured to connect to an outlet of a user's ostomy appliance that is connected about their stoma (and collecting waste exiting the stoma). The ostomy collection device 10 is particularly suited to urostomy use where the waste being collected is more fluid that other ostomy waste outputs and can, therefore, be emptied easily from the collection vessel into a toilet or waste disposal.

The ostomy collection device 10 includes a housing 12, which defines a collecting volume for receiving and storing waste from an ostomy appliance. The ostomy collection device 10 also includes an inlet 30, an outlet 32 and a lid portion 40 (as shown in detail in FIGS. 2 to 4).

The inlet 30 is configured to connect to an outlet of an ostomy appliance (not shown), so that waste from the ostomy appliance flows into the collecting volume. The outlet 32 is configured for emptying the contents of the collecting volume (e.g. into a toilet, etc. so that the waste can be disposed of).

The lid portion 40 is slidable between a first position and a second position. In the first position, the outlet 32 is open and waste can flow through the outlet 32 from the collecting volume (and, thus, waste can be emptied from the collecting volume). In the second position, the lid portion 40 is in a position in which the waste flow is impeded (i.e. the outlet is covered/closed).

In some embodiments, the inlet 30 is aligned with a portion of the outlet 32 when the lid portion 40 is in its second position, which means the outlet 32 is not completely closed. However, usually, the inlet 30 will be connected to a delivery tube which is connectable/connected to the ostomy appliance of the user—even if the ostomy collection device 10 is knocked over, the contents will not spill easily unless the outlet 32 is properly opened by the lid portion 40 (i.e. unless the lid portion 40 is in its first position).

In some embodiments, the inlet 30 is positioned on a different portion/part of the lid portion 40 and the inlet 30 and outlet 32 do not align when the lid portion 40 is moved to its second position. In this case the outlet is closed when the lid portion 40 is in its second position. However, the collecting volume may still be open due to the inlet 30 opening.

In some embodiments, the inlet 30 may include a valve part. The valve part may be opened by the connector from the tube of an ostomy appliance and otherwise biased to a closed condition. Thus, irrespective of where the inlet 30 is positioned, unless that connector is connected to the inlet 30, the inlet 30 is closed. This means that even if the inlet 30 is positioned on the lid portion 40, the second position of the lid portion 40 may close the outlet 32.

The housing 12 has a base 14, a wall 16 and a top 18. In embodiments, the base 14 provides a flat bottom portion to the ostomy collection device 10. The base 14 may be substantially circular (see FIG. 1) but it should be appreciated that this is not necessarily the case.

In embodiments, the wall 16 is connected to the base 14 (i.e. the wall 16 is connected around a peripheral edge of the base 14). The wall 16 extends substantially upwards from the base 14. In the illustrated example, there is a single continuous wall 16, which extends around the entire periphery of the base 14 to create an internal volume defined by the base 14 and the wall 16. It should be appreciated that the wall could be constructed from multiple portions connected together, if desired.

In some embodiments, the wall 16 is ridged or concertinaed which allows the wall 16 to fold upon itself. This results in a collection device 10 that can be collapsed into a small configuration (for example, for travel).

The top 18 of the housing 12 closes the collecting volume. In the illustrated embodiments, the inlet 30 and the outlet 32 are both positioned on the top 18.

Preferably, the top 18 can be removed/disconnected from the wall 16. Thus, in some embodiments, the top 18 and the wall 16 both include respective engagement formations, which engage with each other so that the top 18 connects to the wall 16.

In some embodiments, the engagement formations are corresponding threaded portions. In other words, the top 18 has an engagement formation which incorporates a helical thread and the wall 16 also has an engagement formation which may also be a helical thread. On relative rotation of the two helical threads together, the top 18 and the wall 16 are connected together.

The outlet 32 is located in a recess or channel 42 formed in the top 18 and the lid portion 40 slides along the recess 42. The lid portion 40 is received in the recess 42 and provides a closure over the outlet 32. The lid portion 40 slides along the channel 42 between its first and second positions.

The recess 42, in the illustrated example (FIG. 4), extends across substantially the entire top 18. In other words, the recess 42 extends from one side of the top 18 to the opposing side.

In some embodiments (see FIGS. 2-4, for example), the top 18 includes a stepped portion/flange 18a and a raised part 18b. The stepped portion 18a provides a threaded formation on an interior surface of the top 18, so that it can connect to a corresponding threaded formation at the top of the wall 16. The stepped portion 18a also provides a groove/receiving part that receives a handle 60 (discussed in more detail below).

The lid portion 40 extends across the entire raised part 18b of the top 18. The channel/recess 42 extends from one side of the top 18 to an opposing side and the lid portion 40 extends across the top 18 (in the recess 42). The ends of the lid portion 40 align with the top 18 when the lid portion 40 is in its second (closed) position and the ends move out of alignment with a peripheral edge of the top when in any other position.

In some embodiments, the ostomy collection device 10 further includes a user graspable handle 60. The handle 60 is pivotable between a first position (shown in FIG. 2), which is a “stowed”/storage positon, and a second position (in which the handle 60 is upright and able to be used to carry the device 10).

In the first position in this example, the handle 60 is located on the stepped portion 18a of the top 18. The handle 60, therefore, sits flush with the top surface of the ostomy collection device 10 (i.e. the handle 60 lies substantially flat or in line with the top 18 of the housing).

In its second position, the handle 60 is moved out of alignment with the top 18 to a useable position (i.e. position in which the user can carry the device 10 by the handle 60). The shape of the top 18 may be selected so that a “stop” formation is provided, so that the handle 60 can be positioned upright (away from the top 18) and be supported there stably.

In some embodiments, a locking mechanism is included to restrict and/or inhibit the lid portion 40 from moving out of its second (closed) condition. In the illustrated example, the handle 60 provides a locking mechanism when is its stowed position by preventing the lid portion 40 from moving out of the second position.

The lid portion 40 is an elongate member which slides along a recess in the top 18 (using a rail and projection arrangement, between the top 18 and the lid portion 40, to ensure only reciprocating movement along the channel and no movement in any other direction). When the lid portion 40 is in the closed position, it has only one degree of freedom to move towards the open position. The handle 60 pivots between the stowed position and “in use” position about a pivot point when the handle connects to the top 18 (in this example, the handle is curved and forms a semi-circle—this allows the handle to fit around the raised part of the top 18 in the stowed position). This means that the handle 60 prevents the lid portion 40 from sliding towards the open position (which is the only direction of movement that is available).

In some embodiments, the lid portion 40 includes a movement aid or user graspable portion which assists a user to grasp the lid portion 40 and move it to the desired position. The movement aid may include an additional handle part, a textured surface or groove placed in a useful location for the user.

Another embodiment of a top 18 is shown in FIG. 5—features that are the same or similar to those that have already been described above are provided with the same reference number and include a prime symbol (e.g. the top 18 becomes the top 18′). Features labelled on the figure may not be described explicitly below and it should be appreciated that the description above applies to this embodiment. The main difference between the top 18′ in FIG. 5 and those in FIGS. 2 to 4 is that the lid portion 40′ includes a handle part 80′, which permits the user to grasp the lid portion 40′ and move it between positions. It should be appreciated that this could be combined with the features already described.

In some embodiments, the housing further includes an air vent arrangement (not illustrated). The air vent arrangement functions to permit gases to escape the collecting volume—this prevents or, at least, reduces pressure build up in the collecting volume that could result in malfunction. The air vent arrangement includes a lower part, which is in communication with the collecting volume and an upper part which communicates through an opening in the top 18 to atmosphere.

The air vent arrangement includes a membrane which is water phobic to prevent/inhibit waste exiting the collecting volume through the air vent opening. The membrane or another part of the air vent arrangement may include a carbon component, which is used to neutralise smells in the gas flowing through the air vent arrangement to atmosphere.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. An ostomy collection device including:
a housing which defines a collecting volume for receiving and storing waste from an ostomy appliance,
an inlet for fluidly connecting to an outlet of an ostomy appliance so that waste from the ostomy appliance flows into the collecting volume,
an outlet for emptying the contents of the collecting volume, and
a lid portion which is slidable between a first position, in which the outlet is open and waste can be emptied from the collecting volume and a second position, in which the outlet is closed.

2. An ostomy collection device according to claim 1 wherein the inlet is positioned on the lid portion.

3. An ostomy collection device according to claim 2 wherein the inlet is positioned such that when the lid portion is in the second position, the inlet is substantially aligned with the outlet.

4. An ostomy collection device according to claim 1 further including a handle portion.

5. An ostomy collection device according to claim 4 wherein the handle portion is moveable between an in use position, where the handle is positioned so as to provide a user graspable portion for carrying the ostomy collection device, and a stowed position.

6. An ostomy collection device according to claim 5 wherein when the handle portion is in the stowed position, it is positioned adjacent an edge of the lid portion and acts as a locking mechanism to hold the lid portion in its second position.

7. An ostomy collection device according to claim 1 wherein the lid portion includes a user graspable portion that aids moving it between first and second positions.

8. An ostomy collection device according to claim 1 wherein the inlet, outlet and lid portion are located on a top of the housing.

9. An ostomy collection device according to claim 8 wherein the top includes a channel which receives the lid portion and the lid portion slides in the channel to move between its first and second positions.

10. An ostomy collection device according to claim 9 wherein the channel extends from one side of the top to an opposing side and the lid portion extends across the top and slides out of alignment with the peripheral edge of the top when not in the second position.

11. An ostomy collection device according to claim 8 wherein the housing includes a wall which is substantially upstanding and the wall and the top each have engagement formations which engage to connect the top to the wall.
